(19) United States
(12) Patent Application Publication
(10) Pub. No.: US 2024/0032989 A1
(43) Pub. Date: Feb. 1, 2024

(54) MULTI-NEEDLE ABLATION PROBE

(71) Applicant: ONEPASS MEDICAL LTD., Katzrin (IL)

(72) Inventor: Jacob BEN ARIE, Haifa (IL)

(73) Assignee: ONEPASS MEDICAL LTD., Katzrin (IL)

(21) Appl. No.: 18/265,761

(22) PCT Filed: Dec. 9, 2021
(86) PCT No.: PCT/IL2021/051476

§ 371 Date: Jun. 7, 2023

Related U.S. Application Data
(60) Provisional application No. 63/123,545, filed on Dec. 10, 2020.

(51) Int. Cl. A61B 18/14; A61B 10/02

(52) U.S. Cl. CPC A61B 18/1477; A61B 18/1492; A61B 10/0233; A61B 2018/1467; A61B 2018/143; A61B 2018/00577

(57) ABSTRACT

An ablation device, the device comprising: a sheath; a plurality of needle electrodes that are each extendible from a distal end of the sheath; and a controller that is configured to selectively apply a power signal between specified pairs of needle electrodes of the plurality of needle electrodes, when the plurality of needle electrodes are in contact with a tissue, so as to ablate a desired region of said tissue.

### CROSS REFERENCE TO RELATED APPLICATIONS

This application claims the benefit of priority of U.S. Provisional Patent Application No. 63/123,545 filed Dec. 10, 2020, entitled “MULTI-NEEDLE ABLATION PROBE”, the content of which is incorporated herein by reference in its entirety.

### FIELD OF THE INVENTION

Some embodiments of the present invention relate in general to tissue treatment. More specifically, some embodiments of the present invention relate to tissue ablation using electrical power provided via needle electrode.

### BACKGROUND

In situations where abnormal tissue is present in a patient's body, ablation therapy may be used, e.g., as a substitute for surgical removal, in order to destroy the abnormal tissue. For example, ablation procedure may be used to destroy or ablate a small amount of heart tissue that's causing abnormal heart rhythms, or to treat a tumor in the lung, breast, thyroid, liver, or other area of the body.

Typically, such ablation therapy is less invasive than a surgical procedure to remove the abnormal tissue. For example, with ablation therapy, a probe may be inserted through an incision in the skin, through an artery via a catheter, or by direction of energy beams, e.g., guided by various medical imaging devices. The energy that is applied to ablate the abnormal tissue may include heat (e.g., delivered as radiofrequency or microwave radiation), extreme cold, ultrasound, lasers, or chemicals.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

There is provided, in an embodiment, an ablation device, the device comprising: a sheath; a plurality of needle electrodes that are each extendible from a distal end of the sheath; and a controller that is configured to selectively apply a power signal between specified pairs of needle electrodes of the plurality of needle electrodes, when the plurality of needle electrodes are in contact with a tissue, so as to ablate a desired region of said tissue.

There is also provided, in an embodiment, an ablation method comprising: providing an ablation device comprising: a sheath, a plurality of needle electrodes that are each extendible from a distal end of the sheath, and a controller that is configured to selectively apply a power signal between any pair of needle electrodes of the plurality of needle electrodes; inserting at least a distal portion of said sheath into the body of a subject; extending said plurality of needle electrodes from said distal end of said sheath, such that said plurality of needle electrodes engage with a tissue of said subject; and operating said controller to selectively apply said power signal between specified pairs of said plurality of needle electrodes, to ablate a desired region of said tissue.

In some embodiments, the engaging comprises one of: contact with said tissue, and insertion into said tissue.

In some embodiments, the power signal is a radiofrequency (RF) alternating current.

In some embodiments, the applying comprises applying said power signal to each of said specified pairs of needle electrodes simultaneously.

In some embodiments, the applying comprises applying said power signal between said specified pairs of needle electrodes sequentially, in a predetermined sequence of pairs of needle electrodes of said specified pairs of needle electrodes.

In some embodiments, the specified pairs of needle electrodes are selected based on a desired pattern of ablation.

In some embodiments, the desired pattern is determined based, at least in part, on said desired region of said tissue.

In some embodiments, the plurality of needle electrodes comprises a central first needle electrode and at least two second needle electrodes arranged in a predetermined pattern relative to said first needle electrode.

In some embodiments, the predetermined pattern comprises said at least two second needle electrodes arranged in a surrounding pattern relative to said first needle electrode.

In some embodiments, at least one of said specified pairs of needle electrodes comprises said first needle electrode and one of said second needle electrodes.

In some embodiments, at least one of said specified pairs of needle electrodes comprises a pair of said second needle electrodes.

In some embodiments, the sheath comprises an elongated shaft. In some embodiments, the elongated shaft is flexible.

In some embodiments, a needle electrode of the plurality of needle electrodes comprises an electrical insulation along a length thereof, wherein said needle electrode comprises a non-electrically insulated distal tip. In some embodiments, the non-electrically insulated distal tip is pointed or beveled.

In some embodiments, a needle electrode of the plurality of needle electrodes comprises a sensor. In some embodiments, the sensor comprises a temperature sensor. In some embodiments, the temperature sensor comprises a thermocouple.

In some embodiments, a needle electrode of the plurality of needle electrodes is a biopsy needle comprising a hollow core.

In addition to the exemplary aspects and embodiments described above, further aspects and embodiments will become apparent by reference to the figures and by study of the following detailed description.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. The figures are listed below.

FIG. 1 schematically illustrates a needle electrode ablation device, in accordance with an embodiment of the present invention.

FIG. 2 is a schematically illustrates use of the needle electrode ablation device, in accordance with some embodiments of the present invention;

FIG. 3 schematically illustrates extension of needle electrodes out of a sheath of the needle electrode ablation device, in accordance with some embodiments of the present invention;

FIG. 4A schematically illustrates an example of a control housing of the needle electrode ablation device shown in FIG. 1, in accordance with an embodiment of the present invention, when the needle electrodes are retracted.

FIG. 4B schematically illustrates the control housing of FIG. 4A, with an adapter positioned to shorten a length of the sheath that is insertable into a guide tube.

FIG. 5A schematically illustrates operation of a distal electrode extender slider to extend a first group of needle electrodes out of the sheath.

FIG. 5B schematically illustrates operation of a proximal electrode extender slider to extend a second group of needle electrodes out of the sheath.

FIG. 5C schematically illustrates a proximal end of a control housing of the present device, comprising a proximal port or interface.

FIG. 6A schematically illustrates application of a power signal between a central electrode and selected peripheral electrodes.

FIG. 6B schematically illustrates application of a power signal between selected pairs of adjacent peripheral electrodes.

FIG. 6C schematically illustrates application of a power signal between a central electrode and selected peripheral electrodes, as well as between selected pairs of adjacent peripheral electrodes.

FIG. 6D schematically illustrates application of a power signal between a central electrode and selected peripheral electrodes.

### DETAILED DESCRIPTION

Some embodiments of the present invention provide a tissue ablation device that includes a plurality of needle electrodes that may engage, and/or be inserted into, tissue that is to be ablated.

Typically, the needle electrodes are configured to extend from a distal end of a sheath. The sheath may be in the form of an elongated rigid or flexible shaft that is insertable into a patient's body, e.g., via an incision, or via a guide in the form of a catheter, endoscope tube, or otherwise. The needle electrodes may be retracted into the distal end of the sheath as the needle electrodes are inserted into the patient's body. For example, the retraction into the sheath may avoid contamination of the needle electrodes, as well as snagging or contact, and potential injury to, any anatomical or other structure that is encountered by the sheath as it is inserted into the patient's body. The sheath may also provide electrical insulation between the electrodes and the patient's body or other structure.

In some embodiments, a needle electrode device includes a manually or automatically operated mechanism for remotely extending the needle electrodes out of the sheath. In some embodiments, each needle electrode may be extended individually. Alternatively or in addition, two or more of the needle electrodes may be extended as a group.

A control housing may be located at a proximal end of the device. For example, the control housing may be configured to typically remain outside of the patient's body. The control housing may include a mechanism for extending the needle electrodes out of the sheath, and a controller for selectively applying a power signal to the needle electrodes. In some embodiments, the control housing may include two or more separate housings or units. In some embodiments, at least part of the control housing may function as a handle that may be held and manipulated by an operator of the device.

A controller, which may be at least partially enclosed in the control housing, may be operated to selectively apply a power signal, e.g., a radiofrequency (RF) alternating current, to the needle electrodes, e.g., between any one or more pairs of the needle electrodes. The selective application of the power signal may be in accordance with a predefined sequence of pairs of needle electrodes. For example, the predefined sequence may be designed to substantially uniformly ablate the tissue that is located in a region that is defined by arrangement of contact points of the needle electrodes with the tissue. For example, the region of tissue may be considered to include a region of tissue that is bounded by line segments that connect pairs of the laterally outermost needle electrodes. In the example of a central needle electrode that is located at the center of four outer needle electrodes that are arranged in a square pattern, the region to be ablated may include the tissue that lies within the square region that is defined by contact of the outer needle electrodes with the tissue. Similar regions may be defined for any arrangement of three or more non-collinear needle electrodes.

In some embodiments, the needle electrodes may extend from the proximal control housing, along the length of the sheath, and through a distal end of the sheath. In some embodiments, the controller may be operated to selectively apply a power signal, e.g., RF alternating current, at a proximal end of the needle electrodes, wherein the power signal may be carried by each needle electrode along its length to the distal end thereof.

In some embodiments, the needle electrodes may comprise insulation from a proximal end and along a length thereof, to prevent electrical contact between the portions of the different needle electrodes housed within the sheath. In some embodiments, the electrical insulation extends up to a distal tip portion of the needle electrodes. In some examples, the sheath may include multiple lumens such that each needle electrode extends through a different lumen of the sheath. In this case, the walls that separate between the lumens may provide electrical insulation that prevents electrical contact between the portions of the different needle electrodes that have not been extended out of the distal end of the sheath.

When insertion of the sheath brings a distal end of the sheath to the location of the tissue to be ablated, two or more of the needle electrodes may be extended out of the sheath. The needle electrodes may be extended until their distal ends are in contact with, or are inserted into, the tissue that is to be ablated.

The number of and geometrical arrangement of the needle electrodes may be designed for a particular application or class of applications. For example, a square arrangement of five needle electrodes may include a central electrode that is located approximately equidistantly from four corner electrodes that are arranged at the corners of a square. Other arrangements, e.g., of five needle electrodes, or of fewer than or more than five needle electrodes, may be provided for use as required.

The controller may be configured to verify electrical contact between the needle electrodes and the tissue. For example, the controller may apply a relatively low power signal (e.g., lower than the power that is to be applied during ablation of the tissue) to enable measurement of the electrical impedance between pairs of electrodes. A measured impedance that is less than a threshold impedance may be indicative of sufficient electrical contact to enable ablation.

After electrical contact is formed (and verified) between the needle electrodes and the tissue, a controller of the system may apply a power signal, e.g., e.g., RF alternating current or any other direct current or alternating current between two or more of the needle electrodes in accordance with a predetermined sequence. In some embodiments, the power signal represents an RF alternating current (e.g., in the range of 350-500 kHz).

According to some embodiments, impedance measurement may further be used for identifying location of electrodes, e.g., to identify whether an electrode is located within a blood vessel or in the tissue of the treated subject. In some embodiments, when a controller of the system identifies that one or more electrodes are misplaced, the misplaced electrodes may be deactivated and ablation may be conducted using only properly placed electrodes.

Another use of impedance measurement, according to some embodiments, may be for feedback on the progress of ablation. As may be realized by those skilled in the art, as the ablation process progresses, the impedance of the ablated tissue changes and thus, the change in impedance may be used to determine the progress of the ablation and as a safety measure to prevent undesired damage to the tissue.

The sequence may be selected in accordance with predetermined criteria in order to achieve a desired degree of ablation and/or a desired area coverage of ablation. For example, the sequence may be selected on the basis of impedance measurements, as described above, ultrasound monitoring or other imaging results, or other criteria. The sequence may be empirically determined from laboratory experiments, may be based on simulations or calculations, may be based on a combination of empirical results and calculations, or may be otherwise determined.

In some embodiments, the controller may be configured to selectively apply a power signal, e.g., RF alternating current, between one or more specified pairs of the needle electrodes. In some embodiments, the controller may be configured to selectively apply a power signal between the plurality of specified pairs of the needle electrodes sequentially, in a predetermined sequence of pairs of needle electrodes. In some embodiments, the sequence of application may be selected to form a desired pattern, wherein the desired pattern may be determined based, at least in part, on a desired region of tissue.

In some embodiments, the needle electrodes may comprise a central first needle electrode and at least two second needle electrodes arranged in a predetermined pattern relative to the first needle electrode. In some embodiments, the predetermined pattern comprises the at least two second needle electrodes being arranged in a surrounding pattern relative to the first needle electrode.

In some embodiments, the controller may be configured to selectively apply a power signal, e.g., RF alternating current, between a plurality of specified pairs of the needle electrodes in a predetermined sequence of pairs of needle electrodes, wherein the specified pairs of needle electrodes comprise the first needle electrode and one of the second needle electrodes. In some embodiments, at least one of the specified pairs of needle electrodes comprises a pair of the second needle electrodes. Thus, in the example of the square arrangement of needle electrodes described above, a power signal may be applied between one or more of the corner electrodes and the central electrode, between two adjacent corner electrodes, or between another pair of electrodes. A power signal may be applied sequentially to different pairs or groups of the needle electrodes.

For example, the controller may include circuitry that is configured to selectively and controllably apply a power signal, e.g., RF alternating current, to the needle electrodes. Electrical power for applying the power signal may be generated within the controller, or may be derived from a mains voltage or external power source (e.g., a generator, storage battery, or other electrical power source).

In some embodiments, one or more of the needle electrodes may be curved. As a curved needle electrode is extended from the sheath, the curvature of the needle electrode may cause a lateral distance between the distal end of the needle electrode and an axis that extends from the distal end of the sheath to change. For example, if the needle electrode is curved outward from the axis, extension of that needle electrode out of the sheath increases the lateral distance between the distal end of the electrode and the axis. On the other hand, if the needle electrode is curved inward, toward the axis, extending the needle electrode is out of the sheath decreases the lateral distance between the distal end of the electrode and the axis.

Application of the power signal to the needle electrodes may include, in the example of a central needle electrode that is surrounded by a pattern of outer needle electrodes, sequentially applying power signal to each pairing of the central needle electrode with one or more of the outer needle electrodes. Another example may include sequentially applying power signal to each pair of adjacent outer needle electrodes. Other patterns of applied power signal, e.g., configured for a particular arrangement of needle electrodes, may be employed.

In order to restrict application of the power signal to the region of tissue that is to be ablated, each needle electrode may be electrically insulated along its length except for an exposed, non-insulated distal tip region. In other examples, electrical insulation may be provided by separating walls within the sheath. The tip may be beveled or pointed in order to enable insertion of the expose tip into the tissue that is to be ablated.

One or more of the needle electrodes may be provided with one or more sensors that are configured to sense one or more properties of the tissue, e.g., as the tissue is ablated. For example, the exposed tip of the needle electrode may include a temperature sensor, e.g., in the form of a thermocouple or other type of thermometer, in order to sense the temperature of the tissue. Other types of sensors, e.g., configured to sense one or more mechanical, thermal, chemical, optical, or other property of the tissue, may be included in a needle electrode or elsewhere in a needle electrode ablation device. In some cases, other methods, such as ultrasound measurements, may be applied in order to monitor temperature, elastic modulus, or another property of the tissue prior to, during, or after ablation.

The controller may be configured to monitor the sensed temperature or other property of the tissue. In some cases, the controller may be configured to control application of the power signal in response to a sensed property. For example, application of the power signal may be stopped, paused, or reduced when the sensed temperature exceeds a predefined temperature limit, or resumed when the sensed temperature falls below the temperature limit. The controller may be configured to otherwise control application of power signal in response to a sensed physical or chemical property of the tissue or the surrounding environment.

In some embodiments, the needle electrodes may be in the form of biopsy needles configured to incise tissue samples, which are then aspirated, e.g., using vacuum assist, wherein application of suction to a proximal end of the needle electrodes may draw incised tissue (e.g., into which a pointed distal end of the needle electrode is embedded) into a hollow core of the needle electrode. Accordingly, in some embodiments, the present device may be used alternately or concurrently as a tissue biopsy device and as an ablation device.

In some embodiments, the needle electrodes represent elongated thin leads or cannulas extending from a proximal end of the control housing to a distal end of a sheath. In some embodiments, a proximal end of the needle electrodes may be accessible through a proximal port or terminal or interface, e.g., at the control housing of the device. In some embodiments, in cases where the present device may be used alternately or concurrently as a tissue biopsy device and as an ablation device, the proximal port or interface may also be used as an aspiration port, wherein vacuum suction may be applied to the proximal end of the hollow-core needle electrodes.

In some embodiments, as noted above, the needle electrodes may be insulated along most of their length, save for an exposed distal tip region, so as to prevent contact between any of the needle electrodes within the lumen of the sheath. Accordingly, in some embodiments, electric power may be supplied to the needle electrodes at the proximal port or interface, using, e.g., electrical connections extending from the proximal ends of the needle electrodes to a power source. In some embodiments, the electric power supplied to the needle electrodes may be carried distally along the length of the needle electrodes and to the tip thereof, for performing an ablation procedures as fully detailed herein. In some embodiments, the electric power source may be any suitable power source configured to generate and deliver an RF signal, e.g., an ablation generator.

In some embodiments, a controller module of the present device may be implemented in an add-on module which may be attachable to the proximal port or interface, and provide for electrical connections with each of the proximal ends of the needle electrodes. The add-on module may include the circuitry that is required to apply a power signal, e.g., RF alternating current, to the needle electrodes in order to ablate the tissue.

After electrical contact is formed (and verified) between the needle electrodes and the tissue, a controller of the system may apply a power signal, e.g., RF alternating current or any direct current or alternating current between two or more of the needle electrodes in accordance with a predetermined sequence. In some embodiments, the power signal represents a radiofrequency alternating current (e.g., in the range of 350-500 kHz).

The sequence may be selected in accordance with predetermined criteria in order to achieve a desired degree of ablation and/or a desired area coverage of ablation. For example, the sequence may be selected on the basis of impedance measurements, as described above, ultrasound monitoring or other imaging results, or other criteria. The sequence may be empirically determined from laboratory experiments, may be based on simulations or calculations, may be based on a combination of empirical results and calculations, or may be otherwise determined.

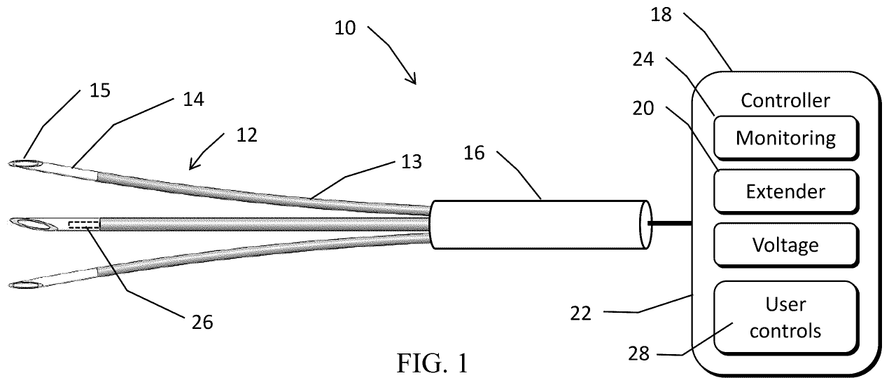

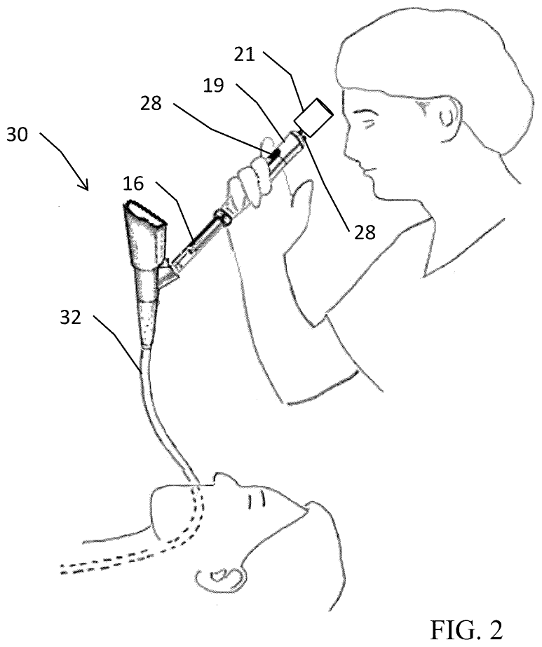

FIG. 1 schematically illustrates a needle electrode ablation device, in accordance with an embodiment of the present invention. FIG. 2 schematically illustrates use of the needle electrode ablation device, in accordance with some embodiments of the present invention;

Needle electrode ablation device 10 is configured to place a plurality of needle electrodes 12 into contact with tissue that is to be ablated, and to apply a power signal to needle electrodes 12 to ablate the tissue. Needle electrodes 12 are extendible from, and retractable into, electrode sheath 16. Needle electrode ablation device 10 may be operated using one or more user controls 28, e.g., located on control housing 19 at a proximal end of electrode sheath 16. In some embodiments, at least some user controls 28 may be located in a separate control unit. The separate control unit may include a computer, smartphone, or other device that has been programmed (e.g., by installation of a program or application) with programmed instructions related to needle electrode ablation device 10. The separate control unit may communicate with other components of needle electrode ablation device 10 via a wired or wireless communications channel.

In some embodiments, one or more needle electrodes 12 may be in the form of a biopsy needle that encloses a hollow core that is in fluidic communication with a suction device. Operation of the suction device may draw a tissue sample into the core at a distal end of that needle electrode 12. In such embodiments, an add-on module 21 may be connected to control housing of a multi-needle biopsy system. Add-on module 21 may include electrical circuitry that is configured to apply a controlled power signal to two or more needle electrodes 12.

At least some components of controller 18 may be located within control housing 19. In some cases, one or more components of controller 18 may be located outside of control housing 19, e.g., in a separate computer or other housing. For example, components of controller 18 that are located outside of control housing 19 may be interconnected by a wired or wireless connection or communications channel. In some cases, one or more components of controller 18 (e.g., power signal control 22, monitoring module 24, or both) may be located within add-on module 21.

Controller 18 includes an extender control 20 that is configured to extend needle electrodes 12 distally out of electrode sheath 16, and to retract needle electrodes 12 proximally into electrode sheath 16. In some examples, extender control 20 may be configured to extend all needle electrodes 12 in tandem out of electrode sheath 16. In some cases, tandem extension of needle electrodes 12 may extend equal lengths of needle electrodes 12 out of electrode sheath 16. In other cases, tandem extension of needle electrodes 12 may extend different needle electrodes 12 by different lengths out of electrode sheath 16. Alternatively or in addition, extender control 20 may be configured to control selective or sequential extension of needle electrodes 12 out of electrode sheath 16. For example, the sequence and length of extension may be predetermined, e.g., in accordance with a predetermined protocol, or may be controlled by an operator of needle electrode ablation device 10, e.g., in response to conditions that are determined by one or more medical imaging devices.

In some embodiments, extender control 20 may include manually operated mechanical components, e.g., levers, sliders, knobs, or other mechanical controls that may be operated by an operator who is holding control housing 19. Alternatively or in addition, extender control 20 may include electrically controllable components. The electrically controllable components may include motorized, hydraulic, pneumatic, electromagnetic, or other actuators or mechanisms. In this case, at least some components of extender control 20 may be located outside of, e.g., in wired or wireless communication with, control housing 19.

Power signal control 22 is configured to selectively apply power signal to needle electrodes 12. Typically, power signal control 22 is operated to concurrently apply a power signal, e.g., a radiofrequency alternating current, to two or more needle electrodes 12. Power signal control 22 includes circuitry for selectively applying and regulating power signal that is applied to needle electrodes 12. Power signal control 22 may include a processor that is configured to apply the power signal in a sequence that is determined in accordance with predetermined criteria, e.g., that are stored as programmed instructions in a data storage unit or memory device with which the processor is in communication.

Electrical power for applying power signal to needle electrodes 12 may be provided to power signal control 22 from an electrical mains, from a generator, from a storage battery or other battery that is located within control housing 19 or external to control housing 19, or otherwise. Typically, power signal control 22 is configured to apply a radiofrequency or other alternating current power signal to needle electrodes 12. For example, circuitry of power signal control 22 may include an alternator, a power signal or current regulator, or other components.

According to some embodiments, impedance measurement may further be used for identifying location of electrodes, e.g., to identify whether an electrode is located within a blood vessel or in the tissue of the treated tissue. In some embodiments, when controller 18 of device 10 identifies that one or more electrodes are misplaced, the misplaced electrodes 12 may be deactivated and ablation may be conducted using only properly placed electrodes 12.

Another use of impedance measurement, according to some embodiments, may be for feedback on the progress of ablation. As may be realized by those skilled in the art, as the ablation process progresses, the impedance of the ablated tissue changes and thus, the change in impedance may be used to determine the progress of the ablation and as a safety measure to prevent undesired damage to the tissue.

Typically, electrode sheath 16 is in the form of an elongated shaft. In some cases, e.g., where electrode sheath 16 is to be inserted into a patient's body via a guide tube 32, e.g., a flexible guide tube of an endoscope 30 as in the example shown in FIG. 2, electrode sheath 16 may be in the form of an elongated flexible shaft. In the example shown, the operator of needle electrode ablation device 10 may monitor placement of needle electrodes 12 by viewing via endoscope 30. In other examples, e.g., where the distal end of electrode sheath 16 is to be inserted a short distance into the body via an orifice or incision, electrode sheath 16 may be in the form of a rigid, or semi-rigid, shaft, e.g., in order to facilitate precise manual placement of the distal end by an operator (e.g., physician or other medically trained person, or a robotic system).

A proximal segment of each needle electrode 12 includes an insulated section 13. The electrical insulation of insulated section 13 is configured to prevent electrical contact of insulated section 13 with another needle electrode 12 or with another surface or object, such as tissue that is not to be ablated, with which insulated section 13 comes into contact.

A distal end of each needle electrode 12 includes exposed tip section 14, which lacks insulation. Exposed tip section 14 is configured for placement in contact with tissue that is to be ablated. In some embodiments, a distal end of exposed tip section 14 may include a beveled surface 15, or may be otherwise pointed. A point at the distal end of exposed tip section 14 may facilitate partial insertion of exposed tip section 14 into tissue that is to be ablated, thus improving electrical contact between exposed tip section 14 and the tissue.

A distal end of a needle electrode 12, e.g., exposed tip section 14, may include one or more sensors 26. Sensor 26 may include a thermocouple or other temperature sensor, or another chemical or physical property of tissue that may be modified by electrical current that passes through the tissue due to application of power signal to needle electrodes 12. Monitoring module 24 of controller 18 may be configured to receive electrical or other signals that are produced by one or more sensors 26. Monitoring module 24 may be configured to analyze signals that are received from a sensor 26 and convert the signals to an indication of a property, e.g., temperature, pH, electrical conductivity, or other property of the tissue or of a surrounding environment.

In some embodiments, power signal control 22 may be configured to adjust a power signal that is applied to needle electrodes 12 in accordance with a property of the tissue that is measured using one or more sensors 26. For example, power signal control 22 may be configured to reduce, e.g., to zero, a power signal that is applied to needle electrodes 12 when a sensed temperature of the tissue exceeds a predetermined temperature.

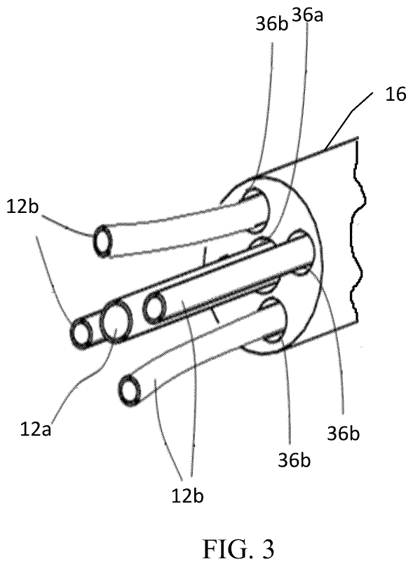

FIG. 3 schematically illustrates extension of needle electrodes out of a sheath of the needle electrode ablation device, in accordance with some embodiments of the present invention;

In the example shown, a central electrode 12a is extendible out of central opening 36a at a distal end of a central lumen of electrode sheath 16. Central electrode 12a is surrounded by a plurality of peripheral electrodes 12b that are each extendible out of a peripheral opening 36b at the distal end of a peripheral lumen of electrode sheath 16. In this context, the term “surrounded by” should be understood as indicating that central opening 36a lies within a polygon formed by connecting pairs of neighboring peripheral openings 36b by line segments.

Material that separates between the lumens of electrode sheath 16 may provide electrical insulation that prevents contact between the sections of needle electrodes 12 that have not been extended out of electrode sheath 16. In some such cases, electrode sheath 16 may provide all electrical insulation that is required, such that a needle electrode 12 need not include separate insulation (such as of insulated section 13).

In the example shown, four peripheral electrodes are arranged in an equally spaced square arrangement about central electrode 12a. In other examples, needle electrodes 12 and openings of electrode sheath 16 may number more or less than four, and may be otherwise arranged.

At least a distal end of one or more of needle electrodes 12, e.g., peripheral electrodes 12b in the example shown, or other needle electrodes 12, may be arced when in an unconstrained, relaxed state. Prior to extension out of an opening of electrode sheath 16, e.g., a peripheral opening 36b or another opening of electrode sheath 16, the shape of the arced needle electrode 12 may be constrained to a curvature of electrode sheath 16. Once a distal end of the arced needle electrode 12 is extended out of the distal end of electrode sheath 16, the distal end of the arced needle electrode 12 may no longer be constrained and may return to the curvature of its relaxed state. In this manner, a distance between exposed tip sections 14 of different needle electrodes 12 may depend on the distance through which those exposed tip sections 14 are extended out of electrode sheath 16.

In the example shown, each peripheral electrode 12b when extended out of its peripheral opening 36b is arced laterally outward from central electrode 12a. Thus, the distance between exposed tip section 14 of a peripheral electrode 12b and exposed tip section 14 of central electrode 12a or of another peripheral electrode 12b may increase with the distance through which those exposed tip sections 14 are extended out of electrode sheath 16. Thus, a human or automated operator of extender control 20 may increase the distance between pairs of exposed tip sections 14 of needle electrodes 12 by increasing the distance through which exposed tip section 14 are extended out of electrode sheath 16.

In other examples, one or more arced needle electrodes 12 may be curved in a different direction, e.g., inward toward central electrode 12a, azimuthally (e.g., with a component that is perpendicular to a radial direction from central electrode 12a to that arced needle electrode 12), or otherwise. In other examples, one more of needle electrodes 12 may be straight in a relaxed state, but an opening in electrode sheath 16, e.g., a peripheral opening 36b, may be slanted. Thus, a needle electrode 12 that is extended out of that opening may be slanted in a particular direction. In all such examples, an operator of extender control 20 may at least partially control the relative locations of contact of exposed tip sections 14 of different needle electrodes 12 with tissue that is to be ablated.

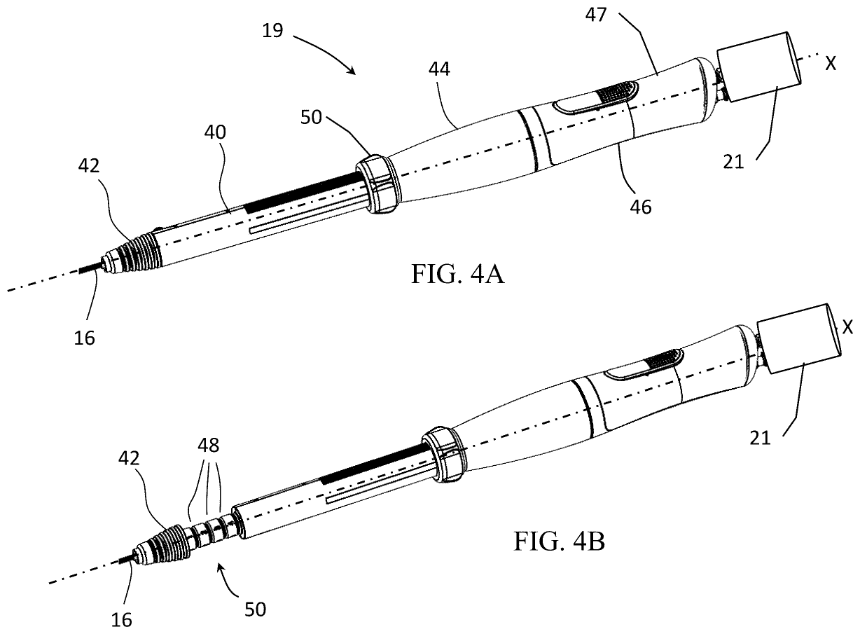

FIG. 4A schematically illustrates an example of a control housing of the needle electrode ablation device shown in FIG. 1, in accordance with an embodiment of the present invention, when the needle electrodes are retracted.

In the example shown, control housing 19 is coupled to the proximal portion of electrode sheath 16. Control housing 19 includes main shaft 40, proximal electrode extender slider 46, and distal electrode extender slider 44 that is configured to slide axially generally along an axis X defined by main shaft 40, relative to housing handle 47. In the example shown, manual operation of components of control housing 19 may selectively extend different groups of needle electrodes 12 out of electrode sheath 16 or retract the groups of needle electrodes 12 into electrode sheath 16.

In some embodiments, control housing 19 includes an adapter 42 located at a distal end of main shaft 40. Adapter 42 may be configured to adapt needle electrode ablation device 10 for insertion of electrode sheath 16 into guide tubes 32, e.g., of an endoscope 30, of varying lengths by adjusting an effective length of sheath 16. For example, adapter 42 may function as a stop that limits insertion of electrode sheath 16 into a guide tube 32.

In the example shown, adapter 42 is configured to slide axially with respect to main shaft in the distal or proximal directions over electrode sheath 16 (e.g., while electrode sheath 16 remains substantially fixed in place) and by that shorten or lengthen, respectively, the length of electrode sheath 16 that is insertable into a guide tube 32.

In the example shown, where adapter 42 is located adjacent to the distal end of main shaft the entire length of electrode sheath 16 that is distal to adapter 42 may be inserted into a guide tube 32. Sliding adapter 42 distally away from main shaft 40 over electrode sheath 16 may shorten the length of electrode sheath 16 that is insertable into guide tube 32.

FIG. 4B schematically illustrates the control housing of FIG. 4B, with an adapter positioned to shorten a length of the sheath that is insertable into a guide tube.

In the example shown, adapter 42 has been slide distally away from main shaft 40, thus shorting the length of electrode sheath 16 that extends distally beyond adapter 42. Sliding adapter 42 proximally back toward main shaft 40 may lengthen the length of electrode sheath 16 that is insertable into guide tube 32.

In some embodiments, control housing 19 is lockable to guide tube 32, e.g., via a Luer-type lock fitting that mates with a counterpart fitting on guide tube 32. Typically, the fitting is coupled to or defined by adapter 42. Typically, a distal end of adapter 42 (shown by example to be generally cone shaped) may be arranged to fit a Luer-lock of endoscope 30 while electrode sheath 16 extends through guide tube 32 in the form of a lumen of endoscope 30 that extends toward the tissue to be ablated within a body of a patient.

In the example shown, adapter 42 is mounted of a distal end of stem 50 that is extendible out of, or insertable into, main shaft 40 to slide adapter 42 distally away from or proximally toward main shaft 40. In the example shown, stem 50 is marked with a plurality of markings to assisting in adjusting the length of electrode sheath 16 that is insertable into guide tube 32. In some embodiments, locking structure may be provided to prevent sliding of stem 50 into or out of main shaft 40 once adapter 42 has been slide to a desired position relative to main shaft 40.

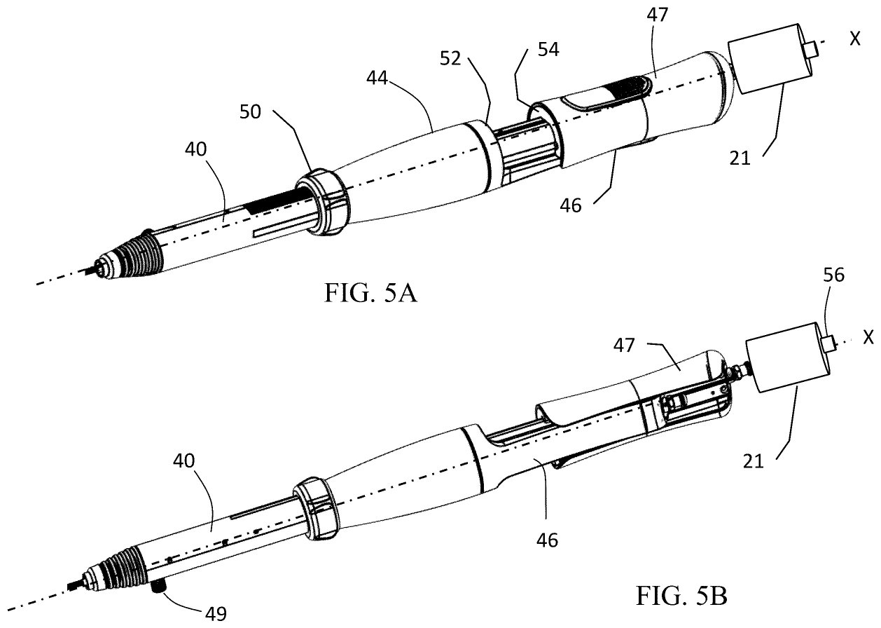

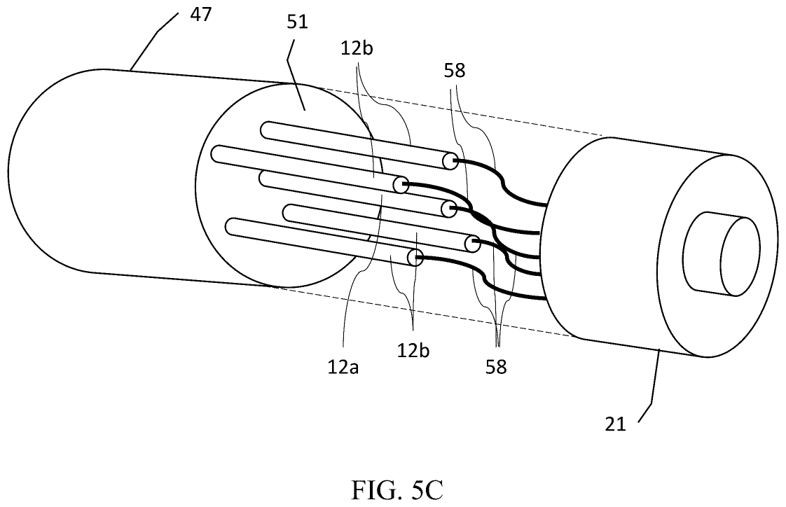

For example, locking structure may include a screw, a spring-loaded pin (e.g., a pogo pin), clip, or other locking structure. For example, locking structure, e.g., spring-loaded pin 49 (visible in FIG. 5B), may be configured to engage one or more stops 48. The stops 48 peripheral slits about a stem 50, as in the example shown, pits, indentations, ridges, or other structure that may be engaged by the locking structure. Stops 48 may also be utilized as indications of pre-set distances to which adapter 42 may be set, e.g., to adapt to a particular type of guide tube 32.

In some embodiments, different groups of needle electrodes 12 may be sequentially extended out of electrode sheath 16 by sequential operation of distal electrode extender slider 44 and proximal electrode extender slider 46.

FIG. 5A schematically illustrates operation of a distal electrode extender slider to extend a first group of needle electrodes out of the sheath.

Distal electrode extender slider 44 is attached to one or more of needles electrode 12 through electrode sheath 16. Thus, sliding of distal electrode extender slider 44 in a distal direction may extend exposed tip section 14 of the needle electrodes 12 that are attached to distal electrode extender slider 44 out of a distal end of electrode sheath 16. In one example, only central electrode 12a is attached to distal electrode extender slider 44. In this manner, central electrode 12a may be inserted into tissue prior to insertion of peripheral electrodes 12b into the tissue. Another group of one or more needle electrodes 12 may be attached to, and may thus be extendible by operation of, distal electrode extender slider 44.

Limiting ring 50 may be slid to a desired location along main shaft 40 and locked into position at that location. Limiting ring 50 may thus limit distal motion of distal electrode extender slider 44, e.g., to limit extension of those needle electrodes 12 that are attached to distal electrode extender slider 44. For example, the limit may ensure that exposed tip section 14 of a control housing 19 does not penetrate a tissue surface by more than a predetermined (e.g., on the basis of medical considerations) distance.

In the example shown, distal electrode extender slider 44 has been distally slid until it contacts limiting ring 50, thus maximally extending the attached needle electrodes 12 to the predetermined limit.

Similarly, sliding of distal electrode extender slider 44 in a proximal direction may retract exposed tip sections 14 of the needle electrodes 12 that are attached to distal electrode extender slider 44 into the distal end of electrode sheath 16.

FIG. 5B schematically illustrates operation of a proximal electrode extender slider to extend a second group of needle electrodes out of the sheath.

In the example shown, proximal electrode extender slider 46 is attached to one or more needle electrodes 12 that are not attached to distal electrode extender slider 44. Thus, distal sliding of proximal electrode extender slider 46 relative to housing handle 47 may extend exposed tip sections 14 of needle electrodes 12 that are attached to proximal electrode extender slider 46 out of the distal end of electrode sheath 16. Distal sliding of proximal electrode extender slider 46 may be limited by contact of distal face 54 of proximal electrode extender slider 46 with proximal face 52 of distal electrode extender slider 44. Similarly, sliding of proximal electrode extender slider 46 in a proximal direction may retract exposed tip sections 14 of the needle electrodes 12 that are attached to proximal electrode extender slider 46 into the distal end of electrode sheath 16.

In some examples, peripheral needles 12b may be attached to proximal electrode extender slider 46. In other examples, another subset of needle electrodes 12 may be attached to proximal electrode extender slider 46. In some examples, all needle electrodes 12 are attached to a single electrode extender slider, or separate controls may be provided to extend each individual needle electrode 12, or other groups of two or more needle electrodes 12.

Alternatively or in addition to a mechanical mechanism for extending needle electrodes 12, needle electrode ablation device 10 may be provided with a motorized, hydraulic, pneumatic, electromagnetic, or otherwise controlled actuator or mechanism for extending all or some of needle electrodes 12 out of electrode sheath 16, or for retracting needle electrodes 12 into electrode sheath 16.

In some embodiments, control housing 19 may be configured to enable an operator of needle electrode ablation device 10 to manipulate control housing 19 and needle electrodes 12 using a single hand.

In some cases, control housing 19, together with any mechanical controls (e.g., distal electrode extender slider 44 and proximal electrode extender slider 46) may be rotatable relative to adapter 42 and to a guide tube 32.

In some cases, electrical power for selectively applying a power signal to needle electrodes 12 may be provided via electrical connection 56. In some cases, one or more of an electrical power supply, control circuitry, user operable controls, or other components may be incorporated into control housing 19 or add-on module 21. When needle electrodes 12 are configured to function also as biopsy needles, a suction source may be applied to needle electrodes 12 via a port on control housing 19 or on add-on module 21.

When needle electrodes 12 are extended, exposed tip sections 14 of two or more needle electrode 12 may contact tissue. In some cases, extension of needle electrodes 12 may cause exposed tip section 14 of one or more of needle electrodes 12 to insert or embed into the tissue surface, e.g., dependent on a distance of extension of. In some cases, a depth to which an exposed tip section 14 is embedded into tissue may be determined by a distance through which adapter 42 is extended distally out of main shaft 40.

When two or more exposed tip sections 14 are in contact with a surface of tissue that is to be ablated, power signal control 22 may be operated to selectively apply a power signal to two or more needle electrodes 12. Application of power signals to needle electrodes 12 may generate an electrical current within the tissue between exposed tip sections 14 to those needle electrodes 12. A sequence of application of power signals selectively to different subsets of needle electrodes 12 may be designed to effectively ablate a region of tissue between exposed tip sections 14 of the needle electrodes 12 of that subset.

FIG. 5C schematically illustrates a proximal end of housing 47 comprising a proximal port or interface 51. Proximal ends of needle electrodes 12a, 12b are provided within port 51. In some embodiments, add-on module 21 may be coupled to proximal end of housing 47, wherein add-on module 21 may be configured to provide power signal to needle electrodes 12a, 12b via electrical connection or leads 58. As noted above, the power signal supplied to the needle electrodes 12a, 12b proximally may be carried distally along the length of the needle electrodes and to the tip thereof, for performing an ablation procedures as fully detailed herein.

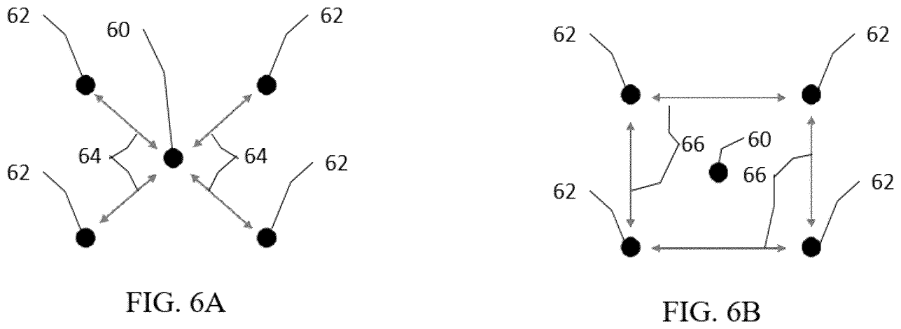

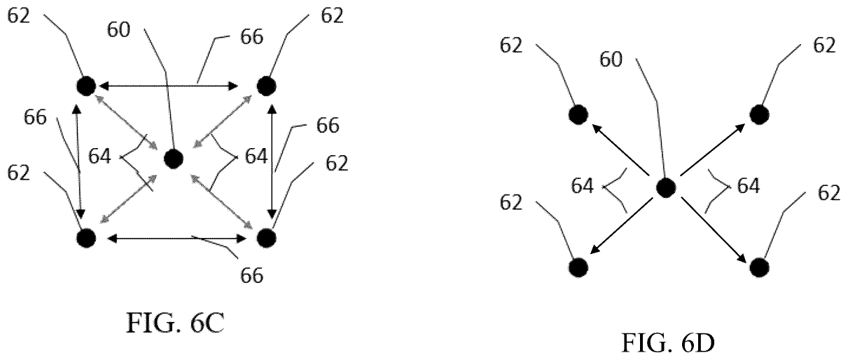

FIG. 6A schematically illustrates application of a power signal between a central electrode and selected peripheral electrodes.

In the example shown, exposed tip section 14 of one electrode, e.g., corresponding to central electrode 12a as shown in FIG. 3, is in contact with a tissue surface at central contact point Similarly, exposed tip sections 14 of four other needle electrodes 12, e.g., corresponding to peripheral electrodes 12b, are shown as in contact with the tissue surface at four peripheral contact points 62, in a square pattern about central contact point 60. In other examples, contact points between a tissue surface and a plurality of exposed tip sections 14 may be otherwise arranged.

A shape that is formed by connecting pairs of adjacent or nearest neighboring peripheral contact points 62 by line segments may substantially define lateral boundaries of a region of the tissue that is to be ablated. A depth to which exposed tip sections 14 of the electrodes are inserted into the tissue may define a thickness of the volume of tissue that is to be ablated.

Each arrow represents an electrical current 64 that may be generated between central contact point 60 and a selected peripheral contact point 62 by application of a power signal to the corresponding needle electrodes 12. Typically, each electrical current 64 is in the form of radiofrequency alternating current. In other examples, direct current, pulsed current, or alternating current having frequency in another frequency range may be generated.

In some cases, power signal control 22 may be configured to generate each electrical current 64 individually, e.g., in a sequence. In some cases, power signal control 22 may be configured to generate two or more of electrical currents 64 concurrently. For example, power signal control 22 may be configured to apply a power signal to all peripheral contact points 62 in tandem, and to central contact point 60 so as to cause the electrical current to flow concurrently between central contact point 60 and all of peripheral contact points 62.

In one embodiment, direct current may be applied concurrently between central contact point 60 and all peripheral contact points 62. For example, central contact point 60 may function as an anode and peripheral contact points 62 may function as cathodes, or vice versa. In other examples, the direct current may be applied sequentially between central contact point 60 and each peripheral contact point 62, or between central contact point 60 and two or more peripheral contact points 62).

FIG. 6B schematically illustrates application of a power signal between selected pairs of adjacent peripheral electrodes.

In the example shown, the arrangement of central contact point 60 and peripheral contact points 62 is the same as in FIG. 6A. Each arrow represents an electrical current 66 that may be caused by power signal control 22 to flow between a pair of adjacent peripheral contact points 62 by application of a power signal to the corresponding needle electrodes 12.

In some cases, each electrical current 66 may be generated sequentially, e.g., by sequential application of a power signal to the corresponding pairs of needle electrodes 12. In some cases, two or more of electrical currents 66 may be generated concurrently. In other examples, a power signal may be applied to other combinations of two or more needle electrodes 12 to generate current between nonadjacent peripheral contact points 62.

In some cases, application of a power signal to selected needle electrodes 12 to generate electrical current 66 between adjacent peripheral contact points 62 may be alternated with generation of electrical current 64 between one or more selected peripheral contact points 62 and central contact point 60.

In other examples, contact points between needle electrodes 12 and a tissue surface may be formed by less or more than five needle electrodes 12. Contact points may be arranged in a pattern other than a pattern of peripheral contact points surrounding a single central contact point.

FIG. 6C schematically illustrates application of a power signal between a central electrode and selected peripheral electrodes, as well as between selected pairs of adjacent peripheral electrodes.

In the example shown, the arrangement of central contact point 60 and peripheral contact points 62 is the same as in FIGS. 6A-6B. Each arrow represents an electrical current 64, 66 that may be caused by power signal control 22 to flow between a central contact point 60 and peripheral contact points 62, and/or a pair of peripheral contact points 62, by application of power signal to the corresponding needle electrodes 12.

The descriptions of the various embodiments of the present invention have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. An ablation device, the device comprising:
a sheath;
a plurality of needle electrodes that are each extendible from a distal end of the sheath; and
a controller that is configured to selectively apply a power signal between specified pairs of needle electrodes of the plurality of needle electrodes, when the plurality of needle electrodes are in contact with a tissue, so as to ablate a desired region of said tissue.

2. The device of claim 1, wherein said power signal is a radiofrequency (RF) alternating current.

3. The device of claim 1, wherein said applying comprises applying said power signal to each of said specified pairs of needle electrodes simultaneously.

4. The device of claim 1, wherein said applying comprises applying said power signal between said specified pairs of needle electrodes sequentially, in a predetermined sequence of pairs of needle electrodes of said specified pairs of needle electrodes.

5. The device of claim 1, wherein said specified pairs of needle electrodes are selected based on a desired pattern of ablation.

6. (canceled)

7. The device of claim 1, wherein said plurality of needle electrodes comprises a central first needle electrode and at least two second needle electrodes arranged in a predetermined pattern relative to said first needle electrode.

8. The device of claim 7, wherein said predetermined pattern comprises said at least two second needle electrodes arranged in a surrounding pattern relative to said first needle electrode.

9. The device of claim 7, wherein at least one of said specified pairs of needle electrodes comprises said first needle electrode and one of said second needle electrodes.

10. The device of claim 7, wherein at least one of said specified pairs of needle electrodes comprises a pair of said second needle electrodes.

11. The device of claim 1, wherein the sheath comprises an elongated shaft.

12. The device of claim 10, wherein the elongated shaft is flexible.

13. (canceled)

14. (canceled)

15. The device of claim 1, wherein a needle electrode of the plurality of needle electrodes comprises a sensor.

16. (canceled)

17. (canceled)

18. The device of claim 1, wherein a needle electrode of the plurality of needle electrodes is a biopsy needle comprising a hollow core.

19. An ablation method comprising:
providing an ablation device comprising:
a sheath;
a plurality of needle electrodes that are each extendible from a distal end of the sheath, and
a controller that is configured to selectively apply a power signal between any pair of needle electrodes of the plurality of needle electrodes;
inserting at least a distal portion of said sheath into the body of a subject,
extending said plurality of needle electrodes from said distal end of said sheath, such that said plurality of needle electrodes engage with a tissue of said subject; and
operating said controller to selectively apply said power signal between specified pairs of said plurality of needle electrodes, to ablate a desired region of said tissue.

20. (canceled)

21. The method of claim 19, wherein said applying comprises applying said power signal to each of said specified pairs of needle electrodes simultaneously.

22. The method of claim 19, wherein said applying comprises applying said power signal between said specified pairs of needle electrodes sequentially, in a predetermined sequence of pairs of needle electrodes of said specified pairs of needle electrodes.

23. (canceled)

24. (canceled)

25. The method of claim 19, wherein said plurality of needle electrodes comprises a central first needle electrode and at least two second needle electrodes arranged in a predetermined pattern relative to said first needle electrode.

26. The method of claim 25, wherein said predetermined pattern comprises said at least two second needle electrodes arranged in a surrounding pattern relative to said first needle electrode.

27. The method of claim 25, wherein at least one of said specified pairs of needle electrodes comprises said first needle electrode and one of said second needle electrodes.

28-37. (canceled)
